(19) United States
(12) Patent Application Publication
(10) Pub. No.: US 2005/0096615 A1
(43) Pub. Date: May 5, 2005

(54) Absorbent article with segmented absorbent structure

(75) Inventors: David A. Kuen, Neenah, WI (US); Seth M. Newlin, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/698,612

(22) Filed: Oct. 31, 2003

(51) Int. Cl. A61F 13/15; A61F 13/20

(52) U.S. Cl. 604/385.01

(57) ABSTRACT

An absorbent article has a substrate and an absorbent structure comprising at least two adjacent absorbent segments arranged in generally edge-facing-edge relationship with each other and having a boundary region therebetween. The absorbent segments are attached to the substrate for movement with the substrate. The absorbent segments and boundary region are constructed to permit movement of the absorbent segments relative to each other at the boundary region therebetween upon movement of the substrate. In other embodiments, the article has at a pair of absorbent structures in superposed relationship between a pair of substrates. Each of the absorbent structures is made up of two or more absorbent segments having a boundary region therebetween, with the absorbent segments of one absorbent structure being free to move relative to the absorbent segments of the other absorbent structure.

### BACKGROUND OF THE INVENTION

The present invention relates generally to absorbent articles intended for personal wear, and more particularly to such absorbent articles having one or more segmented absorbent structures.

Absorbent articles such as diapers, training pants, incontinence garments, etc. conventionally comprise a liquid permeable body-facing liner, a liquid impermeable outer cover, and an absorbent core (also referred to as an absorbent body or absorbent structure) formed separately from the outer cover and liner and disposed therebetween for taking in and retaining liquid (e.g., urine) exuded by the wearer. In some of these absorbent articles, the outer cover and/or the liner are stretchable to permit some expansion of the article when necessary to provide a better fit on the wearer. During use, the article is subjected to forces such as those generated by loading of the absorbent article and movement of the wearer. These forces can cause the absorbent core to shift within the absorbent article, to tear, or to otherwise become permanently distorted, all of which reduce the intended absorbent characteristics of the absorbent core and increase the possibility of liquid body exudates leaking from the article.

To this end, it is known to secure the separately formed absorbent structure to the outer cover and/or the liner, such as using adhesive or by thermal or ultrasonic bonding, to prevent the absorbent core from shifting as the article is stretched and retracted during usage. However, securing the absorbent core to the outer cover or liner in this manner tends to reduce the stretchability of the substrate to which the absorbent core is secured, thereby reducing the flexibility of the absorbent article. Moreover, while securing the absorbent core to the outer cover and/or liner reduces the risk of shifting and distortion, stretching of the substrate to which the separately formed absorbent core is secured can still result in tearing of the absorbent core.

Also, conventional absorbent cores are typically constructed of superabsorbent material particles and hydrophilic fibers loosely mixed and entangled together to form an absorbent batt. In some absorbent cores, the superabsorbent material is concentrated in certain target areas of the absorbent article where more absorption is needed. When such an absorbent core is disposed between a stretchable outer cover and liner, the superabsorbent particles and hydrophilic fibers shift around as the absorbent article is stretched and retracted during usage. The displacement of the superabsorbent particles and hydrophilic fibers from the target areas can lead to leakage from the absorbent article and/or, where the superabsorbent particles accumulate in a certain area, swelling of the particles upon absorbing liquid can make wearing the article feel uncomfortable.

### SUMMARY OF THE INVENTION

In general, an absorbent article according to one embodiment of the present invention comprises a first substrate and a second substrate in generally superposed relationship with the first substrate. A first absorbent structure is disposed between the first and second substrates and comprises at least two absorbent segments arranged in generally adjacent, edge-facing-edge relationship with each other and having a boundary region between the adjacent absorbent segments. A second absorbent structure is also disposed between the first and second substrates in generally superposed relationship with the first absorbent structure and has at least two absorbent segments arranged in generally adjacent, edge-facing-edge relationship with each other and having a boundary region between the adjacent absorbent segments. At least one absorbent segment of the second absorbent structure is in superposed relationship with at least a portion of at least two adjacent absorbent segments of the first absorbent structure and the boundary region between the at least two adjacent absorbent segments of the first absorbent structure.

In another embodiment, the absorbent article generally comprises a first substrate and a second substrate in generally superposed relationship with the first substrate. A first absorbent structure is disposed between the first and second substrates and comprises at least two adjacent absorbent segments arranged in generally edge-facing-edge relationship with each other and having a boundary region therebetween. Each of the absorbent segments of the first absorbent structure is attached to the first substrate for movement therewith. A second absorbent structure is disposed between the first and second substrates in generally superposed relationship with the first absorbent structure and comprises at least two adjacent absorbent segments arranged in generally edge-facing-edge relationship with each other and having a boundary region therebetween. Each of the absorbent segments of the second absorbent structure is attached to the second substrate for movement with the second substrate. The absorbent segments of the second absorbent structure are free from attachment to the absorbent segments of the first absorbent structure to permit movement of the absorbent segments of the second absorbent structure relative to the absorbent segments of the first absorbent structure upon movement of the first and second substrates.

In yet another embodiment, the absorbent article generally comprises a substrate and an absorbent structure comprising at least two adjacent absorbent segments arranged in generally edge-facing-edge relationship with each other and having a boundary region therebetween. The absorbent segments are attached to the substrate for movement with the substrate. The absorbent segments and boundary region are constructed to permit movement of the absorbent segments relative to each other at the boundary region therebetween upon movement of the substrate.

Other features of the invention will be in part apparent and in part pointed out hereinafter.

### BRIEF DESCRIPTION OF THE DRAWINGS

FIG. 1 is a side perspective of an absorbent article of the present invention shown in the form of a pair of training pants having a mechanical fastening system fastened on one side of the training pants and unfastened on the opposite side thereof;

FIG. 2 is a bottom plan view of the training pants of FIG. 1 with the pants in an unfastened, unfolded and laid flat condition, and showing the surface of the training pants that faces away from the wearer;

FIG. 3 is a top plan view similar to FIG. 2 showing the surface of the training pants that faces the wearer when worn and with portions cut away to reveal underlying features;

FIG. 4A is a top plan view similar to FIG. 3 with containment flaps and elastics omitted and with portions cut away to show internal construction;

FIG. 4B is a schematic cross-section taken in the plane of line 4B-4B of FIG. 4A;

FIG. 4C is a schematic cross-section similar to FIG. 4B with the pants in a laterally stretched condition;

FIG. 5 is a top plan view similar to FIG. 4A showing a second embodiment of an absorbent article of the present invention;

FIG. 6 is a top plan view similar to FIG. 4A showing a third embodiment of an absorbent article of the present invention, with containment flaps, elastics and a bodyside liner omitted and portions cut away to reveal underlying construction;

FIG. 7 is a top plan view similar to FIG. 6 showing a fourth embodiment of an absorbent article of the present invention;

FIG. 8 is a top plan view similar to FIG. 6 showing a fifth embodiment of an absorbent article of the present invention;

FIG. 9 is a top plan view similar to FIG. 6 showing a sixth embodiment of an absorbent article of the present invention;

FIG. 10 is a schematic cross-section taken in the plane of line 10-10 of FIG. 9;

FIG. 11 is a schematic cross-section of an outer absorbent structure of FIG. 10 with the absorbent structure shown in an intermediate stage of forming; and

FIG. 12 is a schematic cross-section similar to FIG. 4B but showing an alternative embodiment of an absorbent segment for an absorbent article of the present invention.

Corresponding reference characters indicate corresponding parts throughout the several views of the drawings.

Definitions

Within the context of this specification, each term or phrase below includes the following meaning or meanings:
“Attached” refers to the joining, adhering, connecting, bonding, or the like, of two elements. Two elements will be considered to be attached to one another when they are attached directly to one another or indirectly to one another, such as when each is directly attached to intermediate elements.
“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.
“Layer” when used in the singular can have the dual meaning of a single element or a plurality of elements.
“Liquid impermeable,” when used in describing a layer or multi-layer laminate means that liquid body waste, such as urine, will not pass through the layer or laminate, under ordinary use conditions, in a direction generally perpendicular to the plane of the layer or laminate at the point of liquid contact.
“Liquid permeable” refers to any material that is not liquid impermeable.
“Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

The term “microfibers” means small-diameter fibers having an average diameter not greater than about 100 microns, for example, having a diameter of from about 0.5 microns to about 50 microns, more specifically microfibers may also have an average diameter of from about 1 micron to about 20 microns. Microfibers having an average diameter of about 3 microns or less are commonly referred to as ultra-fine microfibers. A description of an exemplary process of making ultra-fine microfibers may be found in, for example, U.S. Pat. No. 5,213,881, entitled “A Nonwoven Web With Improved Barrier Properties”.

“Non-woven” as used in reference to a material, web or fabric refers to such a material, web or fabric having a structure of individual fibers or threads that are interlaid, but not in a regular or identifiable manner as in a knitted fabric. Non-woven materials, fabrics or webs have been formed from many processes such as, for example, meltblowing processes, spunbonding processes, air laying processes, and bonded carded web processes. The basis weight of non-wovens is usually expressed in ounces of material per square yard (osy) or grams per square meter (gsm) and the fiber diameters are usually expressed in microns. (Note: to convert from osy to gsm, multiply osy by 33.91.).

“Spunbonded fibers”, or “spundbond fibers”, means small-diameter fibers that are typically formed by extruding molten thermoplastic material as filaments from a plurality of fine capillaries of a spinneret having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Petersen, and U.S. Pat. 3,542,615 to Dobo et al., each of which is incorporated by reference in its entirety and in a manner consistent with the present document. Spunbond fibers are quenched and generally not tacky when they are deposited onto a collecting surface. Spunbond fibers are generally continuous and often have average diameters larger than about 7 microns, and more particularly between about 10 and 30 microns. A spunbond material, layer, or substrate comprises spunbonded (or spunbond) fibers.

“Thermoplastic” describes a material which softens when exposed to heat and which substantially returns to a non-softened condition when cooled to room temperature.

### DETAILED DESCRIPTION

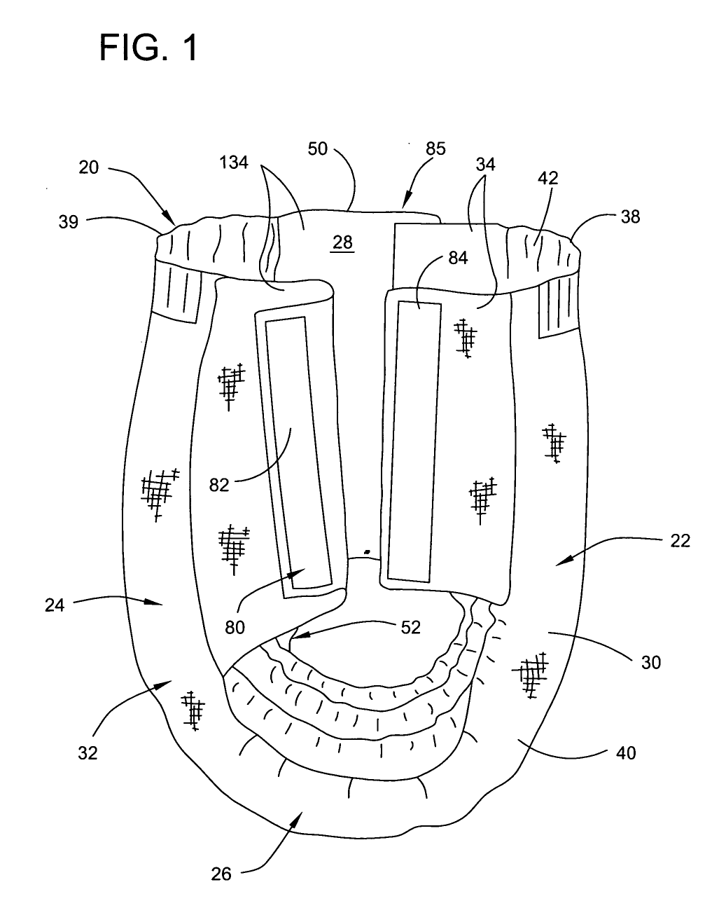

Referring now to the drawings and in particular to FIG. 1, one embodiment of an absorbent article of the present invention is illustrated herein in the form of children's toilet training pants and indicated in its entirety by the reference numeral 20. The absorbent article is suitably, although not necessarily, disposable, which refers to articles that are intended to be discarded after a limited period of use instead of being laundered or otherwise conditioned for reuse. It is understood that the present invention is suitable for use with various other absorbent articles (e.g., other than the pants 20) intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of FIG. 1 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al., each of which is incorporated herein by reference.

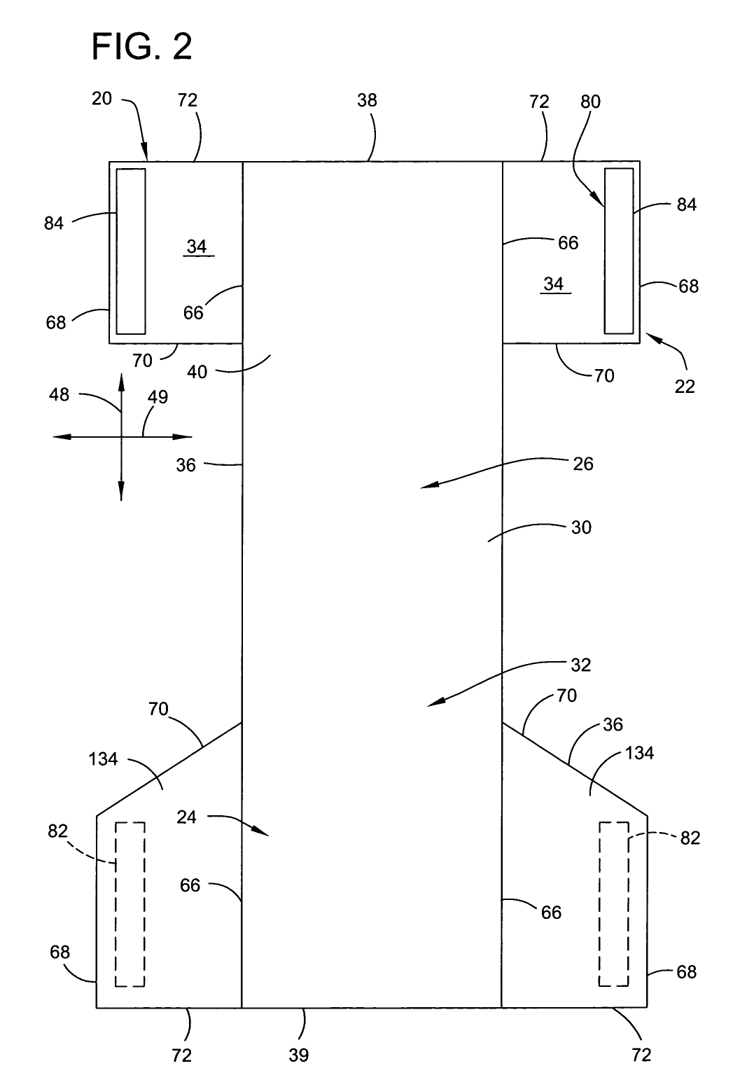

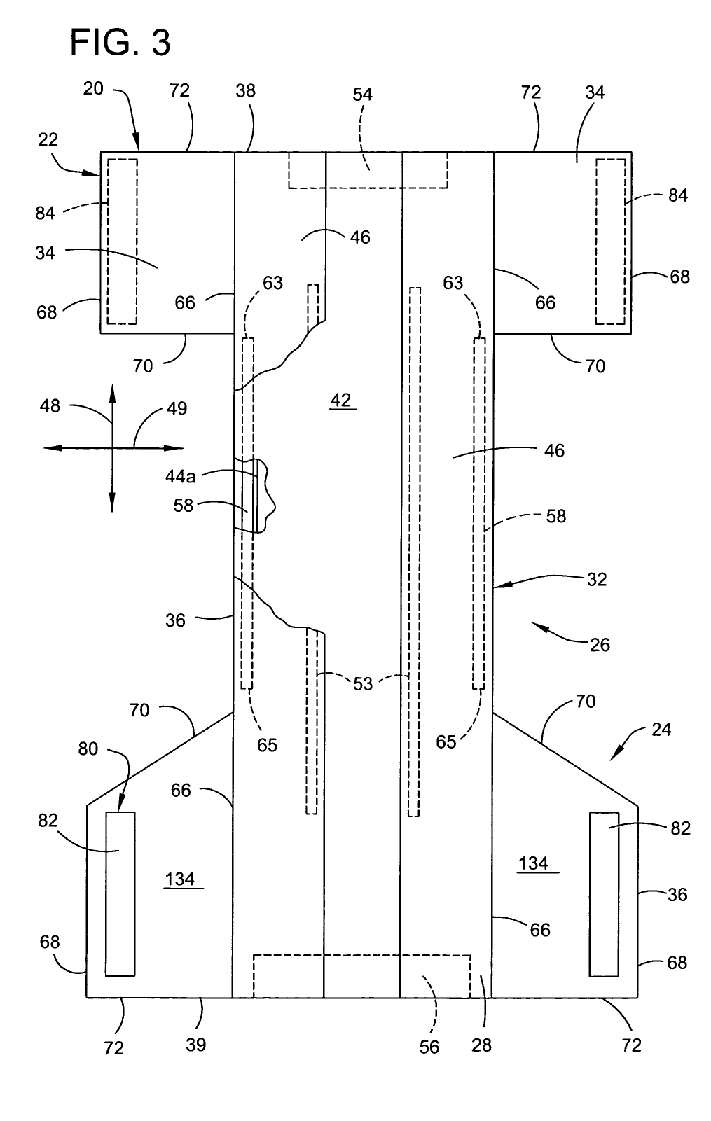

The pair of training pants 20 is illustrated in FIG. 1 in a partially fastened condition and comprises longitudinal end regions, further referred to herein as a front waist region 22 and a back waist region 24, and a center region, further referred to herein as a crotch region 26, extending longitudinally between and interconnecting the front and back waist regions. The pants 20 also has an inner surface 28 which faces the wearer and an outer surface 30 which faces away from the wearer. The front and back waist regions 22, 24 comprise those portions of the pants 20 which, during wear, wholly or partially cover or encircle the waist or mid-lower torso of the wearer. The crotch region 26 generally comprises that portion of the pants 20 which, during wear, is positioned between the legs of the wearer and covers the lower torso and crotch of the wearer. With additional reference to FIGS. 2 and 3, the pair of training pants 20 has (for reference purposes) a longitudinal direction indicated by arrow 48 and a transverse or lateral direction indicated by arrow 49. The pants 20 also has laterally opposite side edges 36 and longitudinally opposite waist edges, respectively designated front waist edge 38 and back waist edge 39.

The illustrated pants 20 comprises a central absorbent assembly, generally indicated at 32, a pair of laterally opposite front side panels 34 extending outward therefrom at the front waist region 22 and a pair of laterally opposite back side panels 134 extending outward therefrom at the back waist region 24. The central absorbent assembly 32 of the illustrated embodiment is generally rectangular. However, it is contemplated that the central absorbent assembly 32 may be other than rectangular, such as hourglass shaped, T-shaped, I-shaped, and the like without departing from the scope of this invention.

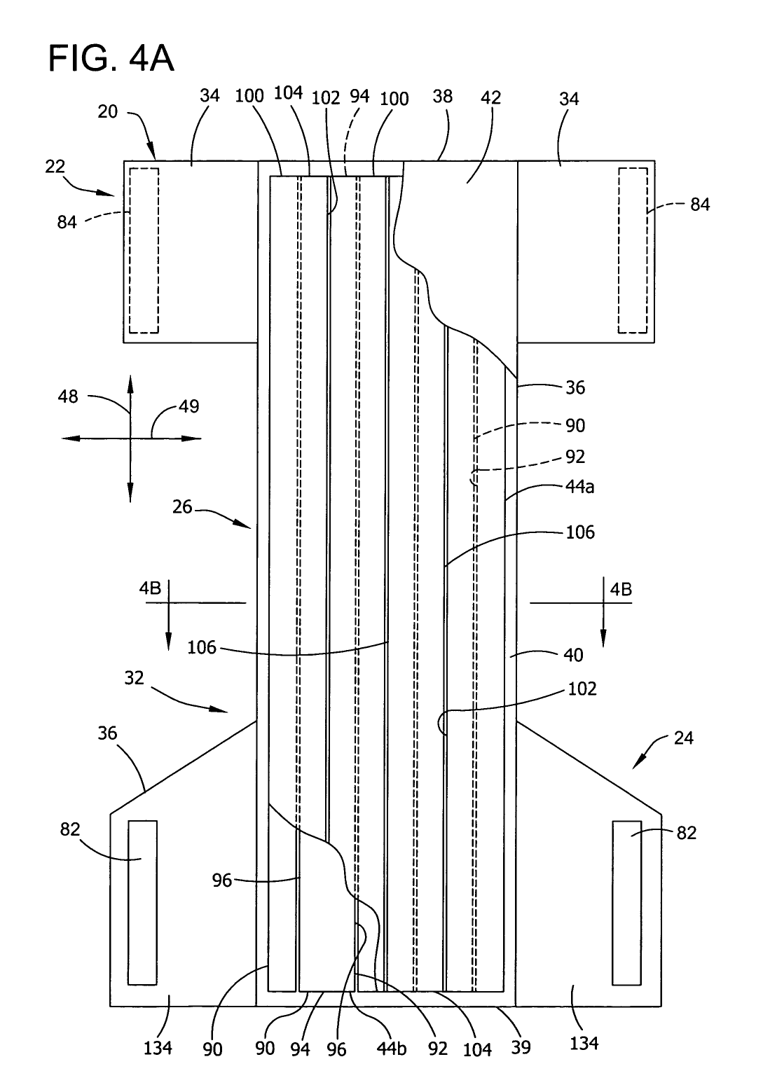

Still referring to FIGS. 1-3, the central absorbent assembly 32 comprises an outer cover 40 (broadly referred to herein as a substrate) and a bodyside liner 42 (FIGS. 1 and 3) (also broadly referred to herein as a substrate) arranged in generally superposed relationship with the outer cover. The liner 42 is suitably adapted (i.e., positioned relative to the other components of the pants 20) for contiguous relationship with the wearer's skin when the pants are worn. The absorbent assembly 32 further comprises a pair of absorbent structures 44a , 44b (FIGS. 4A and 4B) disposed between the outer cover 40 and the bodyside liner 42 for absorbing liquid body exudates released by the wearer. A pair of containment flaps 46 (FIG. 3) is secured to the bodyside liner 42 in laterally spaced relationship with each other to inhibit the transverse flow of body exudates on the liner to the side edges 36 of the absorbent assembly 32. Longitudinally opposite ends of the central absorbent assembly 32 of the illustrated embodiment respectively form portions of the front and back waist edges 38 and 39 of the pants 20, and laterally opposite side edges of the absorbent assembly form portions of the side edges 36 of the training pants.

The outer cover 40 is substantially liquid impermeable to inhibit body exudates against leaking from the pants 20 and wetting articles, such as bed sheets and clothing, as well as the wearer and caregiver. In one embodiment, the outer cover 40 is suitably a multi-layered laminate structure in which at least one of the layers is liquid impermeable. For instance, the outer cover 40 can include a liquid permeable outer layer and a liquid impermeable inner layer affixed together by a laminate adhesive, or by ultrasonic bonds, thermal bonds, or the like. In such an embodiment, the inner layer of the outer cover 40 can be both liquid and vapor impermeable, or it may be liquid impermeable and vapor permeable. For example, the inner layer can be manufactured from a thin plastic film, although other flexible liquid impermeable materials may be used. Alternatively, the outer cover may be constructed of a single layer of material that is liquid impermeable.

Alternative constructions of the outer cover 40 may comprise a woven or non-woven fibrous web layer which has been totally or partially constructed or treated to impart the desired levels of liquid impermeability to selected regions that are adjacent or proximate the absorbent structure. For example, the outer cover may include a gas-permeable, non-woven fabric layer laminated to a polymer film layer which may or may not be gas-permeable. Other examples of fibrous, cloth-like outer cover materials can comprise a stretch thinned or stretch thermal laminate (STL) material.

In a particularly suitable embodiment, the outer cover 40 is stretchable, and even more suitably the outer cover is elastic. As used herein, the term “stretchable” refers to a material that may be extensible and/or elastic. That is, the material may be extended, deformed or the like, without breaking, and may or may not significantly retract after removal of an extending force. The term “elastic” refers to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a significant retractive force.

As an example, the outer cover 40 may comprises a single layer, multiple layers, laminates, spunbond fabrics, films, meltblown fabrics, elastic netting, microporous web, bonded carded webs or foams comprised at least in part of elastomeric or polymeric materials. Elastomeric non-woven laminate webs may include a non-woven material joined to one or more gatherable non-woven webs, films, or foams. Stretch bonded laminates (SBL) and neck bonded laminates (NBL) are examples of elastomeric composites. Examples of suitable non-woven materials are spunbond-meltblown fabrics, spunbond-meltblown-spunbond fabrics, spunbond fabrics, or laminates of such fabrics with films, foams, or other non-woven webs.

Suitable elastomeric materials may include cast or blown films, foams, or meltblown fabrics composed of polyethylene, polypropylene, or polyolefin copolymers, as well as combinations thereof. The elastomeric materials may include PEBAX® elastomer (available from AtoChem located in Philadelphia, Pa.), HYTREL® elastomeric polyester (available from E. I. DuPont de Nemours located in Wilmington, Del.), KRATON® elastomer (available from Shell Chemical Company located in Houston, Tex.), or strands of LYCRA® elastomer (available from E. I. DuPont de Nemours located in Wilmington, Del.), or the like, as well as combinations thereof. The outer cover 40 may include materials that have elastomeric properties imparted by a mechanical process, a printing process, a heating process, and/or a chemical treatment. For example, such materials may be apertured, creped, neck-stretched, heat activated, embossed, micro-strained, or a combination thereof.

The bodyside liner 42 presents a body-facing surface which isolates the wearer's skin from liquids retained by the absorbent structures 44a, 44b and is suitably compliant, soft-feeling, and non-irritating to the wearer's skin. Further, the bodyside liner 42 can be less hydrophilic than the absorbent structures 44a, 44b and is sufficiently porous to be liquid permeable, permitting liquid to readily penetrate through its thickness to reach the absorbent structures. A suitable bodyside liner 42 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers.

Various woven and non-woven fabrics can be included in the bodyside liner 42. For example, the bodyside liner 42 may include a meltblown web, a spunbond web, or a bonded-carded-web composed of the desired fibers. The various fabrics can be composed of natural fibers, synthetic fibers or combinations thereof. In particular aspects, the bodyside liner 42 may be comprised of polymer fibers, networks, laminates, liquid permeable films, cellulosic fibers, rayon, water swellable gels, and elastomeric materials, as well as combinations thereof. Suitable materials for the bodyside liner can include meltblown webs, airlaid webs, spunbond webs, or bonded-carded webs of synthetic continuous or discrete polymer fibers and/or natural fibers, a pattern bonded spunbonded web, airlaid web, or bonded carded web, as well as combinations thereof. Suitable polymers can include polypropylene, polyethylene, polyester, and bi-component materials composed of these polyolefins. The bodyside liner may be composed of a substantially hydrophobic material, and the hydrophobic material may optionally be treated with a surfactant or otherwise processed to impart a desired level of wettability and hydrophilicity.

In a particularly suitable embodiment, the bodyside liner 42 is stretchable, and more suitably the liner is elastic, in addition to or instead of the outer cover. For example, the stretchable bodyside liner 42 can include elastic strands, LYCRA® elastics, cast or blown elastic films, non-woven elastic webs, meltblown or spunbond elastomeric fibrous webs, as well as combinations thereof. Examples of suitable elastomeric materials include KRATON® elastomers, HYTREL® elastomers, ESTANE® elastomeric polyurethanes (available from B.F. Goodrich and Company located in Cleveland, Ohio), or PEBAX® elastomers. It is also understood that both the liner 42 and the outer cover 40 may be non-stretchable without departing from the scope of this invention.

The bodyside liner 42 and outer cover 40 are suitably attached to one another, for example, by being directly attached to each other such as by affixing the outer cover 40 directly to the liner 42, or by being indirectly attached to each other such as by affixing the bodyside liner to intermediate components of the pants 20 which in turn are affixed to the outer cover. The bodyside liner 42 and the outer cover 40 can, for example, be attached to each other along at least a portion of their periphery by adhesive, by ultrasonic bonding, by thermal bonding or by other suitable attachment techniques known in the art.

The containment flaps 46 each have at least one flap elastic member 53 (FIG. 3) secured thereto along an unattached edge of the flap so that the flaps assume a generally upright configuration in at least the crotch region 26 of the training pants 20 to form a seal against the wearer's body. The containment flaps 46 are suitably located adjacent the side edges 36 of the pants 20, and can extend longitudinally along the entire length of the absorbent assembly 32 or may extend only partially along the length of the absorbent assembly. Suitable constructions and arrangements for the containment flaps 46 are generally well known to those skilled in the art and are described in U.S. Pat. No. 4,704,116 issued Nov. 3, 1987 to Enloe, which is incorporated herein by reference.

The training pants 20 also suitably includes a front waist elastic member 54 (FIG. 3), a rear waist elastic member 56, and leg elastic members 58, as are known to those skilled in the art. The waist elastic members 54 and 56 can be operatively joined to the outer cover 40 and/or the bodyside liner 42 along the opposite waist edges 38 and 39, and can extend laterally across all or only a portion of each waist edge. The leg elastic members 58 can be operatively joined to the outer cover 40 and/or the bodyside liner 42 and extend longitudinally adjacent the opposite side edges 36 generally at the crotch region 26 of the training pants 20. Each leg elastic member 58 has a front terminal point 63 and a back terminal point 65, which represent the longitudinal ends of the elastic gathering caused by the leg elastic members.

The flap elastic members 53, the waist elastic members 54 and 56, and the leg elastic members 58 can be formed of any suitable elastic material. Suitable elastic materials include sheets, strands or ribbons of natural rubber, synthetic rubber, or thermoplastic elastomeric polymers. The elastic materials can be stretched and adhered to a substrate (e.g., such as the outer cover 40 and/or the bodyside liner 42), adhered to a gathered substrate, or adhered to a substrate and then elasticized or shrunk, such as upon the application of heat, so that elastic retractive forces are imparted to the substrate.

The side panels 34, 134 are attached along seams 66 to the central absorbent assembly 32 in the respective front and back waist regions 22 and 24. More particularly, as seen best in FIGS. 2 and 3, the front side panels 34 are permanently attached to and extend transversely outward beyond the side edges of the absorbent assembly 32 at the front waist region 22, and the back side panels 134 are permanently attached to and extend transversely outward beyond the side edges of the absorbent assembly at the back waist region 24. The side panels 34, 134 may be attached to the absorbent assembly 32 using adhesive, or by thermal or ultrasonic bonding, or by other suitable attachment techniques. Alternatively, the side panels 34 and 134 can be formed as an integral portion of another component of the absorbent assembly 32. For example, the side panels can comprise a generally wider portion of the outer cover 40, the bodyside liner 42, and/or another component of the absorbent assembly 32.

The front and back side panels 34, 134 have respective outer edges 68 which broadly define the side edges 36 of the pants 20 at the front and back waist regions 22, 24 thereof. The side panels 34, 134 also have respective leg end edges 70 disposed toward the longitudinal center of the absorbent assembly 32, and respective waist end edges 72 which further define the respective front or back waist edges 38, 39 of the pants 20. The leg end edges 70 of the back side panels 134 are illustrated as being curved and/or angled relative to the transverse direction 49 to provide a better fit of the pants 20 about the wearer's legs. However, it is understood that the leg end edges 70 of the front side panels 34 may additionally, or alternatively, be curved or angled, or none of the leg end edges may be curved or angled, without departing from the scope of this invention.

The side panels 34, 134 suitably, although not necessarily, comprise an elastic material capable of stretching generally in the transverse direction 49. Suitable elastic materials, as well as one process of incorporating elastic side panels into training pants, are described in the following U.S. Pat. No.: 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, suitable elastic materials from which the side panels 34, 134 may be constructed may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference. Alternatively, the side panel material may comprise other woven or non-woven materials, such as those described previously as being suitable for construction of the outer cover 40 and/or the bodyside liner 42; mechanically pre-strained composites; or extensible but non-stretchable (e.g., inelastic) materials.

With the training pants 20 fastened as is partially illustrated in FIG. 1, the front and back side panels 34, 134 are fastened together by a fastening system 80 in a three-dimensional configuration of the pants to defined a waist opening 50 and a pair of leg openings 52 of the pants. In such a configuration, the front and back side panels 34 and 134 constitute those portions of the training pants 20 which are positioned on the hips of the wearer. The front and back waist edges 38 and 39 of the training pants 20 are configured to encircle the waist of the wearer and, together with the side panels 34, 134, define the waist opening 50 (FIG. 1) of the pants. Portions of the side edges 36 of the pants 20 in the crotch region 26, together with the fastened side panels 34, 134, generally define the leg openings 52 of the pants 20.

The fastening system 80 comprises laterally opposite first fastening components 82 adapted for refastenable engagement to corresponding second fastening components 84. In one embodiment, a front or outer surface of each of the fastening components 82, 84 comprises a plurality of engaging elements. The engaging elements of the first fastening components 82 are adapted to repeatedly engage and disengage corresponding engaging elements of the second fastening components 84 to releasably secure the pants 20 in its three-dimensional configuration.

The fastening components 82, 84 can comprise any refastenable fasteners suitable for absorbent articles, such as adhesive fasteners, cohesive fasteners, mechanical fasteners, or the like. In particular embodiments the fastening components 82, 84 comprise mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated embodiment, the first fastening components 82 comprise loop fasteners and the second fastening components 84 comprise complementary hook fasteners. Alternatively, the first fastening components 82 may comprise hook fasteners and the second fastening components 84 may comprise complementary loop fasteners. In another embodiment, the fastening components 82, 84 can comprise interlocking similar surface fasteners, or adhesive and cohesive fastening elements such as an adhesive fastener and an adhesive-receptive landing zone or material; or the like. Although the training pants 20 illustrated in FIG. 1 show the back side panels 134 overlapping the front side panels 34 upon fastening thereto, the training pants can alternatively be configured so that the front side panels overlap the back side panels when attached. One skilled in the art will recognize that the shape, density and polymer composition of the hooks and loops may be selected to obtain the desired level of engagement between the fastening components 82, 84. A more aggressive hook material may comprise a material with a greater average hook height and/or a greater percentage of directionally-aligned hooks. When engaged, the fastening components 82, 84 of the illustrated embodiment define refastenable engagement seams 85 (FIG. 1).

The absorbent structures 44a, 44b (broadly referred to herein as inner and outer absorbent structures, respectively, based on the relative positions of the absorbent structures to the wearer of the pants 20) are suitably arranged in generally superposed relationship with each other between the outer cover 40 and the liner 42, and are more suitably arranged in generally overlapping relationship with each other. Each of the absorbent structures 44a, 44b is suitably compressible, conformable and capable of absorbing and retaining liquid body exudates released by the wearer. The absorbent structures 44a, 44b may be constructed of any of a number of well known materials suitable for taking in and retaining liquid body exudates. For example, in one embodiment the outer absorbent structure 44b is suitably composed of a matrix of hydrophilic fibers and particulate superabsorbent material. The hydrophilic fibers are more suitably cellulosic fluff. One suitable pulp fluff is identified with the trade designation CR1654, commercially available from U.S. Alliance, Childersburg, Ala., U.S.A., and is a bleached, highly absorbent sulfate wood pulp containing primarily soft wood fibers and about 16 percent hardwood fibers. As an alternative to wood pulp fluff, synthetic fibers, polymeric fibers, meltblown fibers, short cut homofil bicomponent synthetic fibers, or other natural fibers may be used without departing from the scope of this invention.

As used herein, the term “superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride. The superabsorbent materials can be selected from natural, synthetic, and modified natural polymers and materials. The superabsorbent materials can be inorganic materials, such as silica gels, or organic compounds, such as cross-linked polymers. The term “cross-linked” refers to any means for effectively rendering normally water-soluble materials substantially water insoluble but swellable. Such means can include, for example, physical entanglement, crystalline domains, covalent bonds, ionic complexes and associations, hydrophilic associations, such as hydrogen bonding, and hydrophobic associations or Van der Waals forces. Examples of suitable synthetic superabsorbent material polymers include the acidic or alkali metal and ammonium salts of poly(acrylic acid) and poly (methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), or basic or chloride and hydroxide salts of polyvinyl amine, polyamine polyquarternary ammonium, polyimine, hydrolyzed polyamide, and mixtures and copolymers thereof.

Other suitable superabsorbent material polymers include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and the like. Mixtures of natural and wholly or partially synthetic absorbent polymers can also be useful in the present invention. Additional suitable superabsorbent materials are disclosed in U.S. Pat. No. 3,901,236 issued Aug. 26, 1975 and processes for preparing synthetic absorbent gelling polymers are disclosed in U.S. Pat. No. 4,076,663 issued Feb. 28, 1978 and U.S. Pat. No. 4,286,082 issued Aug. 25, 1981.

The superabsorbent material used in forming the outer absorbent structure 44b can be of any desired configuration, such as spiral or semi-spiral, cubic, rod-like, polyhedral, random, spherical (e.g., beads), needles, flakes, and fibers. Conglomerates of particles of superabsorbent material may also be used in forming the absorbent structure 44b. The superabsorbent material can be substantially homogeneously mixed with the hydrophilic fibers or non-uniformly mixed therewith. The concentration of the superabsorbent particles can also vary through the thickness of the absorbent structure 44b. As a general rule, the superabsorbent material is present in the outer absorbent structure 44b in an amount up to about 90 weight percent based on total weight of the absorbent assembly 32. Alternatively, the outer absorbent structure 44b can be a laminate of fibrous webs and superabsorbent material, a foam or other suitable absorbent web construction known to those skilled in the art.

In the illustrated embodiment of FIGS. 4A and 4B, the outer absorbent structure 44b more suitably comprises a plurality of absorbent segments 90 (e.g., at least two, and in the illustrated embodiment more than two) arranged in generally edge-facing-edge relationship with each other. Boundary regions 92 (e.g., at least one and in the illustrated embodiment a plurality thereof) separate adjacent absorbent segments 90 of the outer absorbent structure 44b. In the particular embodiment illustrated in FIGS. 4A and 4B, adjacent absorbent segments 90 of the outer absorbent structure 44b are suitably discrete (e.g., detached, or non-interconnected), and are more suitably spaced from each other whereby the boundary regions 92 comprise the spacing between adjacent absorbent segments. It is also contemplated that adjacent absorbent segments 90 may instead be in abutting, edge-facing-edge relationship. For example, when the outer cover 40 is in a relaxed or otherwise non-stretched condition as shown in FIG. 4B, the boundary regions 92, i.e., the spacing between adjacent absorbent segments 90, is suitably less than about 5 millimeters (mm) and is more suitably in the range of 0 to about 3 mm. However, it is understood that the spacing between adjacent absorbent segments 90 of the outer absorbent structure 44b may be greater than about 5 mm without departing from the scope of the invention.

The absorbent segments 90 of the absorbent structure 44b illustrated in the embodiment of FIGS. 4A and 4B are generally elongate and rectangular, having longitudinally opposite ends 94 disposed generally adjacent the front and back waist edges 38, 39 of the absorbent assembly 32. The absorbent segments 90 also have laterally opposite side edges 96 which are disposed in generally edge-facing-edge relationship with a corresponding side edge of at least one adjacent absorbent segment.

The absorbent segments 90 of the outer absorbent structure 44b are attached to the outer cover 40, such as by adhesive, by thermal or ultrasonic bonding or by other suitable attachment technique, within an attachment region 98 (FIG. 4B) which is suitably sized (e.g., in length and/or width) smaller than the absorbent segment. That is, the attachment region 98 does not extend to the longitudinal ends 94 and lateral edges 96 of the absorbent segment 90. By attaching each absorbent segment 90 to the outer cover 40, the absorbent segments generally move with the outer cover, and more particularly where the outer cover is stretchable the absorbent segments separate further from each other at the boundary regions 92 upon stretching of the outer cover, e.g., whereby the spacing between adjacent absorbent segments increases as shown in FIG. 4C. The relatively small attachment regions 98 at which the absorbent segments 90 are attached to the outer cover 40 reduces the amount of stiffness imparted to the outer cover by the attachment and allows more of the outer cover to stretch relative to the absorbent segments.

In one embodiment, the absorbent segments 90 of the outer absorbent structure 44b may be generally constructed by cutting a formed absorbent structure into the discrete absorbent segments. For example, the absorbent structure 44b may be a conventional air-formed absorbent structure. Apparatus and methods for air forming a fibrous absorbent web are well known to those skilled in the art and will not be further described herein except to the extent necessary to set forth the present invention. For example, see U.S. Pat. No. 4,666,647 entitled APPARATUS AND METHOD FOR FORMING A LAID FIBROUS WEB issued May 19, 1987; and U.S. Pat. No. 4,761,258 entitled CONTROLLED FORMATION OF LIGHT AND HEAVY FLUFF ZONES issued Aug. 2, 1988. Other such apparatus are described in U.S. Pat. No. 6,330,735 entitled APPARATUS AND PROCESS FOR FORMING A LAID FIBROUS WEB WITH ENHANCED BASIS WEIGHT CAPABILITY issued Dec. 18, 2001; and U.S. patent application Ser. No. 09/947,128, entitled MULTI-STAGE FORMING DRUM COMMUTATOR filed Sep. 4, 2001. The formed absorbent structure 44b is then cut through its thickness into the desired number and shape of the discrete absorbent segments 90. Alternatively, each absorbent segment 90 may be formed separately and arranged relative to each other on the outer cover 40 in the desired arrangement.

In the illustrated embodiment, the absorbent segments 90 of the outer absorbent structure 44b all have generally the same basis weight, density and thickness. However, it is understood that some or all of the absorbent segments 90 may have different basis weights, densities and/or thicknesses relative to each other. It is also contemplated that the concentration of superabsorbent material may be non-uniform among some or all of the absorbent segments 90. For example, absorbent segments 90 having a higher concentration of superabsorbent material may be placed in a target region such as the crotch region 26 and absorbent segments having a lower concentration of superabsorbent material may be placed toward the front and back waist regions 22, 24. It is also contemplated that the basis weight, density, thickness and/or superabsorbent material concentration within one or more of the absorbent segments 90 may be non-uniform across the width and/or along the length of the absorbent segment itself.

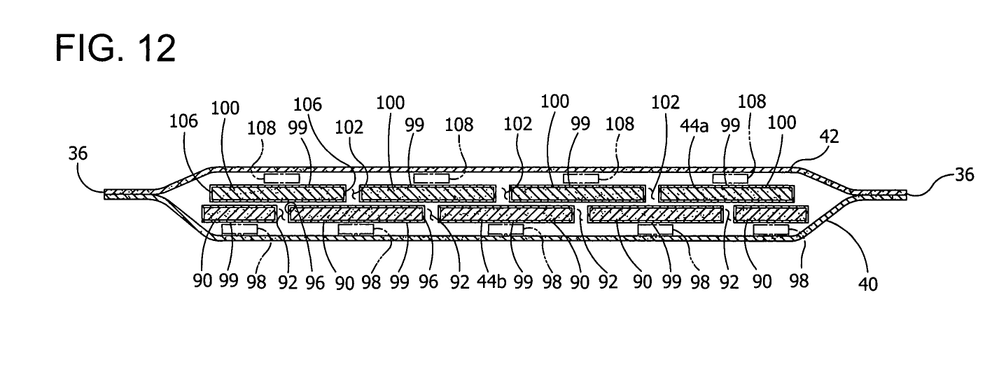

In an alternative embodiment as illustrated in FIG. 12, each absorbent segment 90 may also have a liquid permeable wrapping or enclosure 99, whereby each absorbent segment is in the form of a discrete envelope or packet containing absorbent material, to inhibit absorbent material, such as superabsorbent particles, against migrating away from each respective absorbent segment. The enclosure 99 is suitably sufficiently stretchable to accommodate expansion of the superabsorbent material upon absorbing liquid. Suitable enclosure 99 materials include porous woven materials, porous nonwoven materials (e.g., spunbond and meltblown webs), and apertured films. Examples include, without limitation, any stretchable porous sheet of polymeric fibers, bonded carded webs of synthetic or natural fibers, or combinations thereof. The enclosure 99 material may also be an apertured stretchable plastic film. It is also contemplated that the envelope may be formed by folding a tissue material wherein the absorbent material is disposed within the fold so as to permit expansion swelling of the absorbent material without rupturing the tissue.

A wide variety of polymers may be used to make the enclosure 99 material including without limitation, polyolefins (including polyethylene, polypropylene, and alpha-olefin copolymers thereof); diblock, triblock or multi-block elastomeric copolymers such as olefinic copolymers, including styrene-isoprene-styrene, styrene-butadiene-styrene, styrene-ethylene/butylene-styrene, or styrene-ethylene/propylene-styrene, which may be obtained under the trade designation KRATON® elastomeric resin available from Shell Chemical Company located in Houston, Tex.; polyurethanes, including those available from E. I. Du Pont de Nemours Co., located in Wilmington, Del., under the trade name LYCRA® polyurethane; polyamides, including polyether block amides available from Ato Chemical Company located in Philadelphia, Pa., under the trade name PEBAX® polyether block amide; polyesters, such as those available from E. I. Du Pont de Nemours Co., under the trade name HYTREL® polyester; and single-site or metallocene-catalyzed polyolefins having density less than about 0.89 grams/cc, available from Dow Chemical Co. under the trade name AFFINITY®. The enclosure 99 material may suitably be made of a material having a low coefficient of friction to facilitate absorbent segments 90 of the outer absorbent structure 44b to slide freely relative to the inner absorbent structure 44a upon movement of the outer cover.

Referring again to FIGS. 4A and 4B, the inner absorbent structure 44a is suitably constructed of any of the materials described above from which the outer absorbent structure 44b can be constructed. Moreover, the inner absorbent structure 44a may be constructed from the same materials as the outer absorbent structure 44b, or from different materials without departing from the scope of this invention. In the illustrated embodiment of FIGS. 4A and 4B, the inner absorbent structure 44a comprises a plurality of absorbent segments 100 (e.g., at least two, and in the illustrated embodiment more than two) arranged in generally edge-facing-edge relationship with each other. Boundary regions 102 (e.g., at least one and in the illustrated embodiment a plurality thereof) separate adjacent absorbent segments 100 of the inner absorbent structure 44a.

In the particular embodiment illustrated in FIGS. 4A and 4B, adjacent absorbent segments 100 of the inner absorbent structure 44a are suitably discrete (e.g., detached, or non-interconnected), and are more suitably spaced from each other whereby the boundary regions 102 comprise the spacing between adjacent absorbent segments. It is also contemplated that adjacent absorbent segments 100 may be in abutting, edge-facing-edge relationship. For example, when the liner 42 is a generally relaxed or otherwise non-stretched condition as shown in FIG. 4B, the boundary regions 102, i.e., the spacing between adjacent absorbent segments 100, is suitably less than about 5 millimeters (mm) and is more suitably in the range of 0 to about 3 mm. However, it is understood that the spacing between adjacent absorbent segments 100 of the inner absorbent structure 44a may be greater than about 5 mm. Also, while the spacing between adjacent absorbent segments 100 of the inner absorbent structure 44a is shown in FIG. 4B as being generally the same as the spacing between adjacent absorbent segments 90 of the outer absorbent structure 44b, it is contemplated that the spacing between the absorbent segments of the inner absorbent structure may be greater or less than the spacing between the absorbent segments of the outer absorbent structure without departing from the scope of this invention.

The absorbent segments 100 of the inner absorbent structure 44a illustrated in the embodiment of FIGS. 4A and 4B are generally elongate and rectangular in correspondence with the absorbent segments 90 of the outer absorbent structure 44b. The absorbent segments 100 of the inner absorbent structure 44a are also oriented to extend in the same direction (e.g., longitudinally as shown in FIG. 4A) as the absorbent segments 90 of the outer absorbent structure 44b. More suitably, as seen best in FIG. 4B, the absorbent segments 100 of the inner absorbent structure 44a are arranged relative to the absorbent segments 90 of the outer absorbent structure 44b so that at least one of the absorbent segments 100 of the inner absorbent structure 44a is in overlapping relationship with a portion of at least two adjacent absorbent segments 90 of the outer absorbent structure 44b and the boundary region 92 therebetween. In addition (or alternatively), at least one of the absorbent segments 90 of the outer absorbent structure 44b is in overlapping relationship with at least two adjacent absorbent segments 100 of the inner absorbent structure 44a and the boundary region 102 therebetween. In this manner, all or part of each boundary region 102 between adjacent absorbent segments 100 of the inner absorbent structure 44a is overlaid by one or more absorbent segments 90 of the outer absorbent structure 44b and all or part of each boundary region 92 between adjacent absorbent segments 90 of the outer absorbent structure 44b is overlaid by one or more absorbent segments 100 of the inner absorbent structure 44a.

More particularly, in the illustrated embodiment of FIGS. 4A and 4B the inner absorbent structure 44a and the outer absorbent structure 44b are of generally equal length and width. The inner absorbent structure 44a comprises four longitudinally extending absorbent segments 100 each having the same length and width. The outer absorbent structure 44b comprises five longitudinally extending absorbent segments 90, all having the same length as the absorbent segments 100 of the inner absorbent structure 44a. The three central absorbent segments 90 of the outer absorbent structure 44b have the same width as the absorbent segments 100 of the inner absorbent structure 44a and the laterally outermost absorbent segments of the outer absorbent structure have a width equal to about one-half the width of each of the three central absorbent segments. The spacing between adjacent absorbent segments 90 of the outer absorbent structure 44b is substantially the same as the spacing between adjacent absorbent segments 100 of the inner absorbent structure 44a. In this manner, the total width of the inner absorbent structure 44a is the generally the same as the total width of the outer absorbent structure 44b (and is more specifically slightly less than the total width of outer absorbent structure because the outer absorbent structure comprises one additional boundary region 92). However, the arrangement is such that each of the absorbent segments 100 of the inner absorbent structure 44a overlaps a portion of each of two adjacent absorbent segments 90 of the outer absorbent structure 44b (and the boundary region 92 therebetween). While the laterally outermost absorbent segments 90 of the outer absorbent structure 44b each overlap a portion of only one absorbent segment 100 of the inner absorbent structure 44a, each of the three central absorbent segments 90 of the outer absorbent structure overlaps a portion of each of two adjacent absorbent segments 100 (and the boundary region 102 therebetween) of the inner absorbent structure so that all of the boundary regions 92, 102 between adjacent absorbent segments 90, 100 of the inner and outer absorbent structures 44a, 44b are overlapped by absorbent segments of the opposing absorbent structure.

It is understood that the absorbent segments 100 of the inner absorbent structure 44a may have different lengths and/or widths relative to each other and/or relative to the absorbent segments 90 of the outer absorbent structure 44b. It is also contemplated that the total length and/or width of the inner absorbent structure 44a may be substantially different from the respective length and/or width of the outer absorbent structure 44b and remain within the scope of this invention. In an alternative embodiment (not shown), the absorbent segments 100, 90 of the inner and outer absorbent structures 44a, 44b may be oriented to instead extend laterally across the absorbent assembly 32, or the absorbent segments of one of the inner and outer absorbent structures may be oriented to extend longitudinally of the absorbent assembly while the absorbent segments of the other absorbent structure are oriented to extend other than longitudinally, such as laterally across the absorbent assembly, without departing from the scope of this invention.

The absorbent segments 100 of the inner absorbent structure 44a may be constructed using the same method and device used to construct the absorbent segments 90 of the outer absorbent structure 44b, or they may be constructed in a different manner. For example, the absorbent segments 100 of the inner absorbent structure 44a may be cut from an air-formed absorbent structure, or they may be formed separately from each other. It is also contemplated that a single formed absorbent structure may be folded over upon itself to define opposed inner and outer absorbent structures 44a, 44b and then the opposed structures cut to form the absorbent segments 100, 90. In such an embodiment, the absorbent segments 100, 90 of the inner and outer absorbent structures 44a, 44b are already arranged relative to each other following cutting thereof. Also, the basis weight, density, thickness and/or superabsorbent material concentration of the absorbent segments 100 of the inner absorbent structure 44a may be different from that of the absorbent segments 90 of the outer absorbent structure 44b and remain within the scope of this invention.

The absorbent segments 100 of the inner absorbent structure 44a are suitably attached to the bodyside liner 42, such as by adhesive, by thermal or ultrasonic bonding or by other suitable attachment technique, within an attachment region 108 (FIG. 4B). The attachment region 108 is suitably sized (e.g., in length and/or width) smaller than the absorbent segment 100. That is, the attachment region 108 does not extend to the longitudinal ends 104 and lateral edges 106 of the absorbent segment 100. By attaching each absorbent segment 100 to the liner 42, the absorbent segments of the inner absorbent structure 44a generally move with the liner, and more particularly where the liner is stretchable the absorbent segments separate further from each other upon stretching of the liner, e.g., whereby the spacing between adjacent absorbent segments increases as shown in FIG. 4C. The absorbent segments 100 of the inner absorbent structure 44a are suitably free from any attachment to the absorbent segments 90 of the outer absorbent structure 44b to permit independent movement of the absorbent segments of the inner and outer absorbent structures relative to each other upon stretching of the liner 42 and/or outer cover 40.

In use, e.g., during wear, the pair of training pants 20 is subjected to various forces, such as those caused by initially fitting the pants on the wearer, by walking, sitting, twisting and like, and/or upon loading of the absorbent structures 44a, 44b (e.g., upon absorption of liquid body exudates). In response to these forces, the outer cover 40 and/or bodyside liner 42 stretch (e.g., where the outer cover and/or bodyside liner is stretchable) to facilitate a more comfortable fit of the pants on the wearer. The absorbent segments 90, 100 attached to the stretched outer cover 40 and/or liner 42 further separate from each other at the boundary regions 92, 102 between adjacent absorbent segments as shown in FIG. 4C without damage to the absorbent segments or otherwise shifting thereof within the absorbent assembly 32. By overlapping the boundary regions 102 between adjacent absorbent segments 100 of the inner absorbent structure 44a with the absorbent segments 90 of the outer absorbent structure 44b, and overlapping the boundary regions 92 between adjacent absorbent segments of the outer absorbent structure with the absorbent segments of the inner absorbent structure, liquid body exudates penetrating through the liner 42 flow into at least one of the inner and outer absorbent structures for absorption upon flowing through the thickness of the absorbent assembly 32.

It is contemplated that additional components or layers may be disposed between the bodyside liner 42 and the outer cover 40 generally in contact with one of the absorbent structures 44a, 44b. For example, the pants 20 may further comprise a surge layer (not shown), which may also be broadly referred to as a substrate, disposed between the inner absorbent structure 44a and the bodyside liner 42. Surge layers are generally well known in the art as being constructed to quickly collect and temporarily hold liquid surges, and to transport the temporarily held liquid to the absorbent structures 44a, 44b. It is contemplated that the absorbent segments 100 of the inner absorbent structure 44a may be attached to the surge layer, and the surge layer may be attached to the liner 42, to indirectly attach the absorbent segments to the liner.

Various woven and non-woven fabrics can be used to construct the surge layer. For example, the surge layer may be a layer made of a meltblown or spunbond web of synthetic fibers, such as polyolefin fibers. The surge layer may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer may be composed of a substantially hydrophobic material, and the hydrophobic material may optionally be treated with a surfactant or otherwise processed to impart a desired level of wettability and hydrophilicity.

Additional materials suitable for the surge layer are set forth in U.S. Pat. No. 5,486,166 issued Jan. 23, 1996 in the name of C. Ellis et al. and entitled “FIBROUS NONWOVEN WEB SURGE LAYER FOR PERSONAL CARE ABSORBENT ARTICLES AND THE LIKE”; U.S. Pat. No. 5,490,846 issued Feb. 13, 1996 in the name of Ellis et al. and entitled “IMPROVED SURGE MANAGEMENT FIBROUS NONWOVEN WEB FOR PERSONAL CARE ABSORBENT ARTICLES AND THE LIKE”; and U.S. Patent No. 5,364,382 issued Nov. 15, 1994 in the name of Latimer et al. and entitled “ABSORBENT STRUCTURE HAVING IMPROVED FLUID SURGE MANAGEMENT AND PRODUCT INCORPORATING SAME”, the disclosures of which are hereby incorporated by reference in a manner consistent with the present document.

Also, while two absorbent structures 44a, 44b are shown in the embodiment of FIGS. 4A, 4B and 4C, it is contemplated that more than two absorbent structures may be disposed between the liner 42 and outer cover 40 in superposed relationship, and more suitably overlapping relationship, with each other. In such an embodiment, at least two and more suitably each of the absorbent structures comprises a plurality of absorbent segments constructed and arranged relative to each other in any manner previously or later described herein.

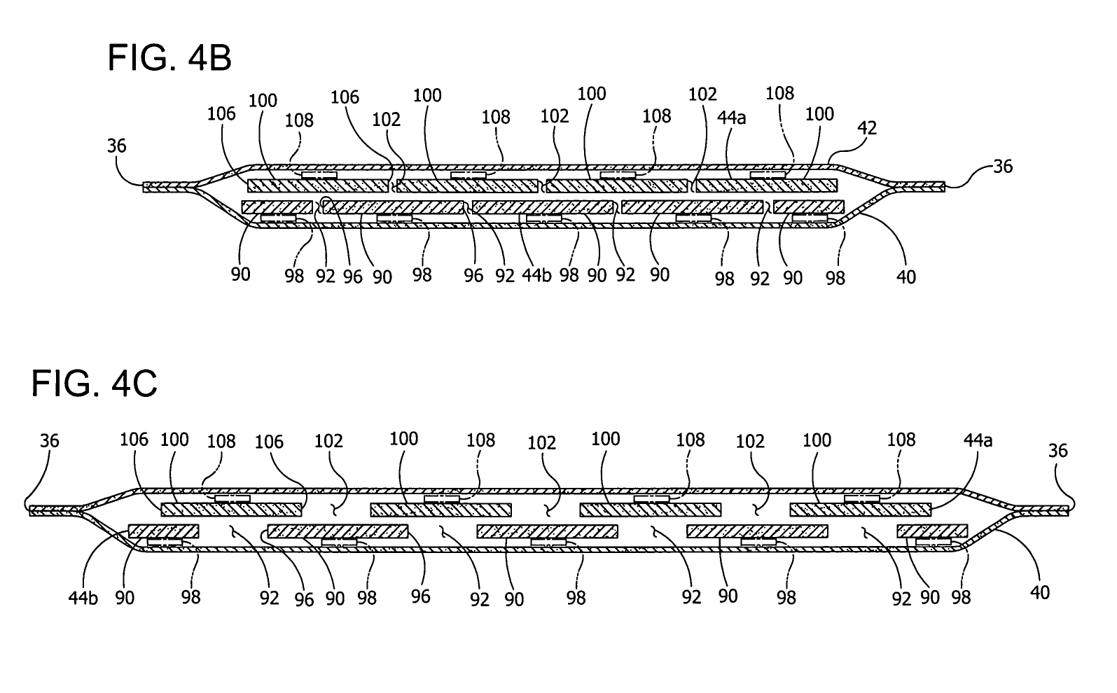

FIGS. 5-8 illustrate additional embodiments of absorbent articles of the present invention, also in the form of training pants, in which inner and outer absorbent structures disposed between the bodyside liner 42 and the outer cover 40 each comprise respective absorbent segments. More specifically, in the illustrated embodiment of FIG. 5, the outer absorbent structure 144b comprises a plurality of elongate absorbent segments 190 arranged in generally edge-facing-edge relationship with each other and having boundary regions 192 therebetween. At the crotch region 26 of the absorbent assembly 32, the absorbent segments 190 of the outer absorbent structure 144b are oriented to extend generally transversely across the absorbent assembly 32. Additional absorbent segments 190 of the outer absorbent structure 144b are disposed at the front waist region 22 of the absorbent assembly 32 and are oriented to extend generally longitudinally from the transversely extending absorbent segments toward the front waist edge 38 of the absorbent assembly. In a like manner, other absorbent segments 190 are disposed at the back waist region 24 of the absorbent assembly 32 and oriented to extend generally longitudinally from the transversely extending absorbent segments toward the back waist edge 39 of the absorbent assembly.

The inner absorbent structure 144a comprises a plurality of elongate absorbent segments 200 arranged in generally edge-facing-edge relationship and having boundary regions 202 therebetween. The absorbent segments 200 are oriented in the respective front waist region 22, the crotch region 26 and the back waist region 24 of the absorbent assembly 32 in the same manner as the absorbent segments 190 of the outer absorbent structure 144b, with at least one of the absorbent segments of the inner absorbent structure overlaying a portion of each of at least two adjacent absorbent segments of the outer absorbent structure and the boundary region 192 therebetween as described previously in connection with the embodiment of FIGS. 4A, 4B and 4C. In addition, or alternatively, at least one of the absorbent segments 190 of the outer absorbent structure 144b overlays a portion of each of at least two adjacent absorbent segments 200 of the inner absorbent structure 144a and the boundary region 202 therebetween as also described previously. The absorbent segments 190 of the outer absorbent structure 144b are suitably attached to the outer cover 40 of the absorbent assembly 32 and the absorbent segments 200 of the inner absorbent structure 144a are suitably attached to the liner 42 of the absorbent assembly. However, it is understood that the absorbent segments 190 may be free from attachment to the outer cover 40 and/or the absorbent segments 200 may be free from attachment to the liner 42 without departing from the scope of this invention.

The orientation of the absorbent segments 200, 190 of the inner and outer absorbent structures 144a, 144b within the front waist region 22, crotch region 26 and back waist region 24 of the absorbent assembly 32 corresponds generally to the various directions in which the training pants 20 typically stretches in order to accommodate the fit of the training pants on the wearer during use. For example, absorbent segments 190, 200 disposed in the crotch region 26 of the absorbent assembly 32 are oriented to extend transversely because the liner 42 and outer cover 40 of the training pants 20 tend to stretch in the longitudinal direction 48 during use. Orienting the absorbent segments 190, 200 transversely in the crotch region 26 permits the absorbent segments to further separate from each other as the outer cover 40 and/or liner 42 stretch in the longitudinal direction 48. The absorbent segments 190, 200 disposed in the front and back waist regions 22, 24 are oriented to extend longitudinally to permit further separation between adjacent absorbent segments upon stretching of the outer cover 40 and/or liner 42 in the transverse direction 49 at the front and back waist regions during use.

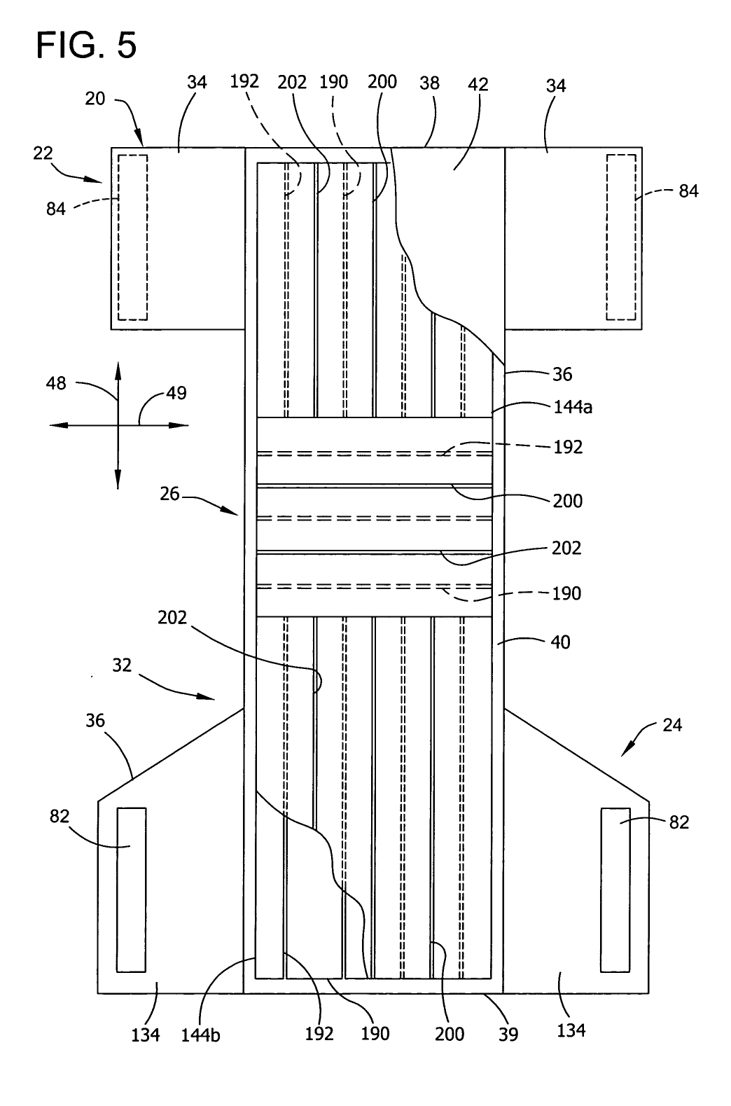

In the embodiment illustrated in FIG. 6, the absorbent segments 300, 290 of the inner and outer absorbent structures 244a, 244b are generally rectangular whereby the boundary regions 302, 292 between adjacent absorbent segments extend both laterally and longitudinally of the absorbent assembly 32 in a generally grid-like pattern. In this manner, at least one absorbent segment 290 of the outer absorbent structure 244b is in overlaid relationship with portions of at least two adjacent absorbent segments 300 of the inner absorbent structure 244a and the boundary region 302 therebetween, and/or at least one absorbent segment of the inner absorbent structure is in overlaid relationship with portions of at least two adjacent absorbent segments of the outer absorbent structure and the boundary therebetween. Other absorbent segments 290, 300 of the outer and inner absorbent structures 244b , 244a are in overlaid relationship with portions of four adjacent absorbent segments of the opposed absorbent structure so that the intersections formed by longitudinally and laterally extending boundary regions 292, 302 are overlaid by the absorbent segments 300, 290.

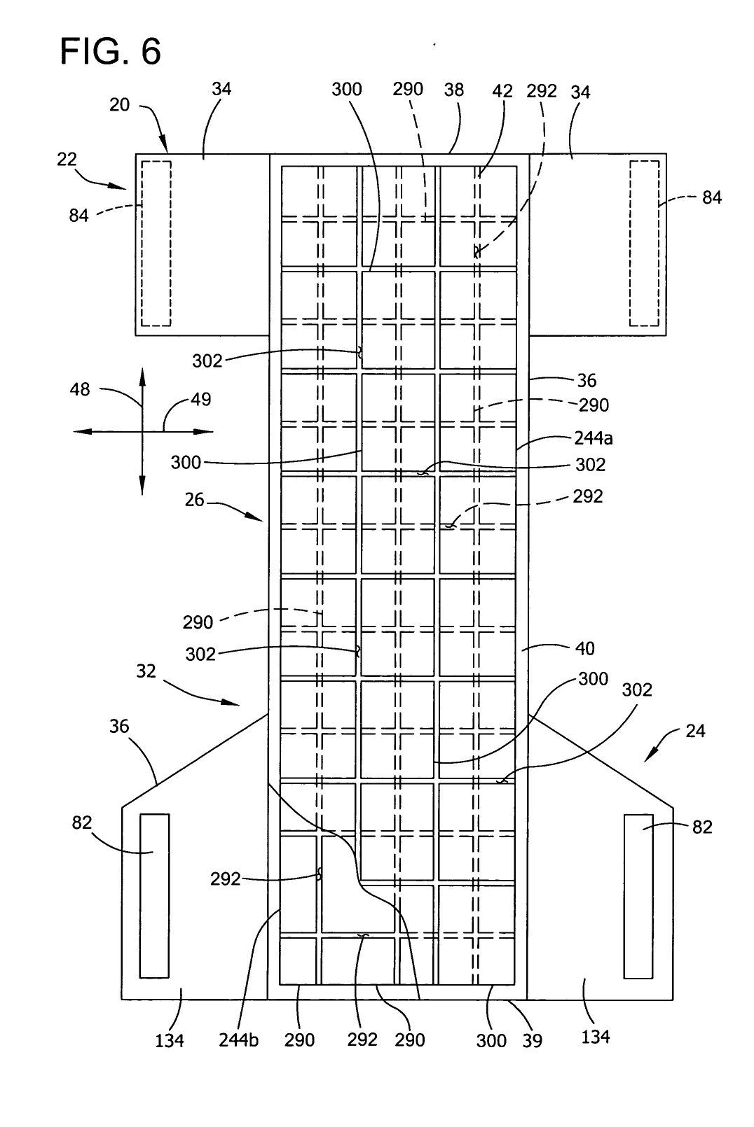

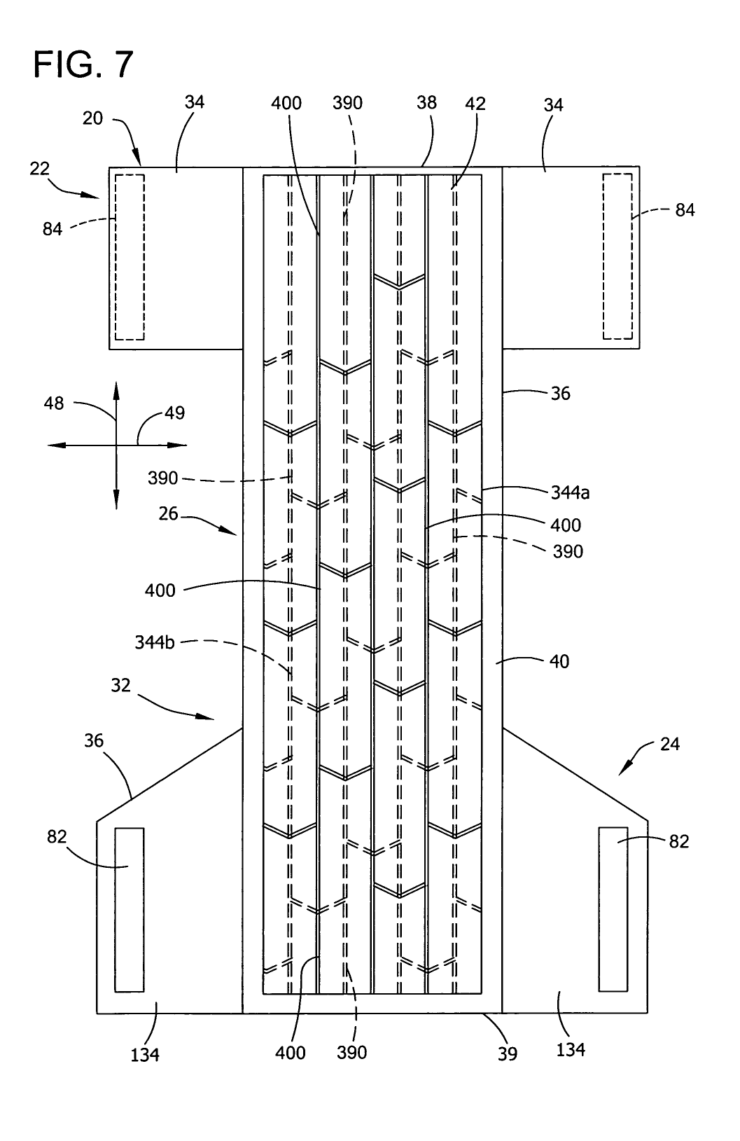

As an example, the absorbent segments 290, 300 may each have a length in the range of about 1 cm to about 5 cm, and more suitably a length of about 2.5 cm. The absorbent segments 290, 300 may have a width in the range of about 1 cm to about 5 cm. It is understood that the absorbent segments 290, 300 may alternatively be square, triangular, diamond shaped, or other suitable shape without departing from the scope of this invention. For example, in the embodiment illustrated in FIG. 7, the absorbent segments 400, 390 of the inner and outer absorbent structures 344a, 344b are each generally elongate and of a generally chevron shape. The absorbent segments 500, 490 of the inner and outer absorbent structures 444a, 444b of the embodiment of FIG. 8 are suitably generally irregular in shape.

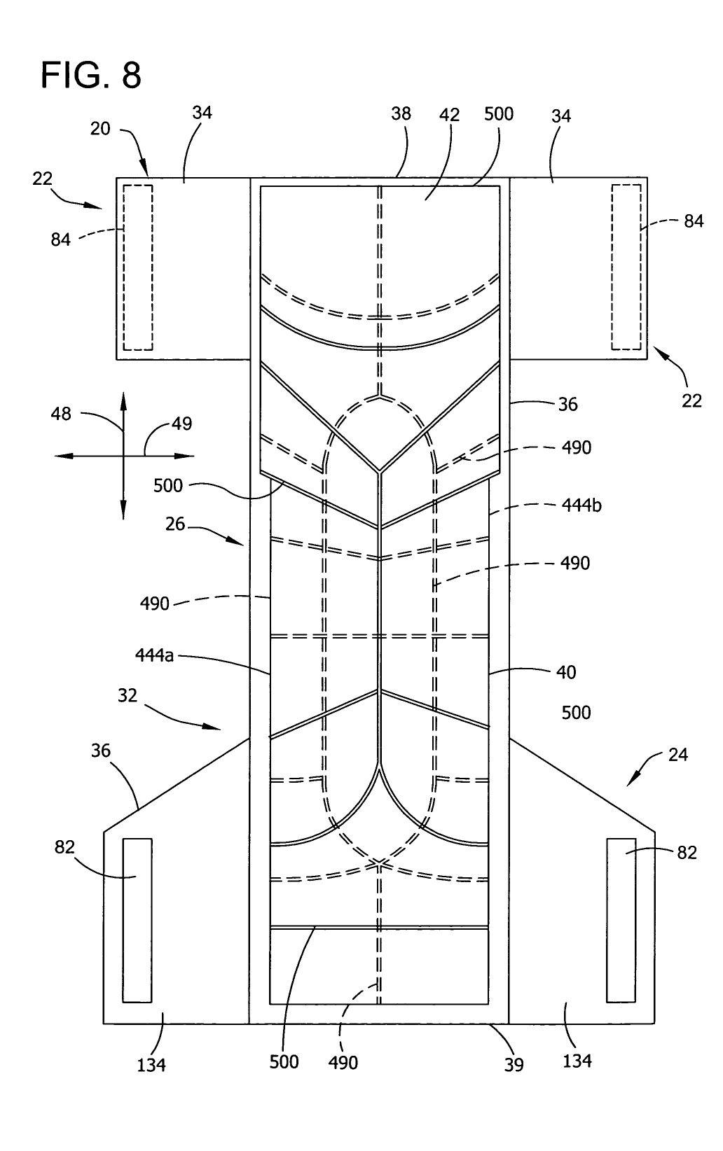

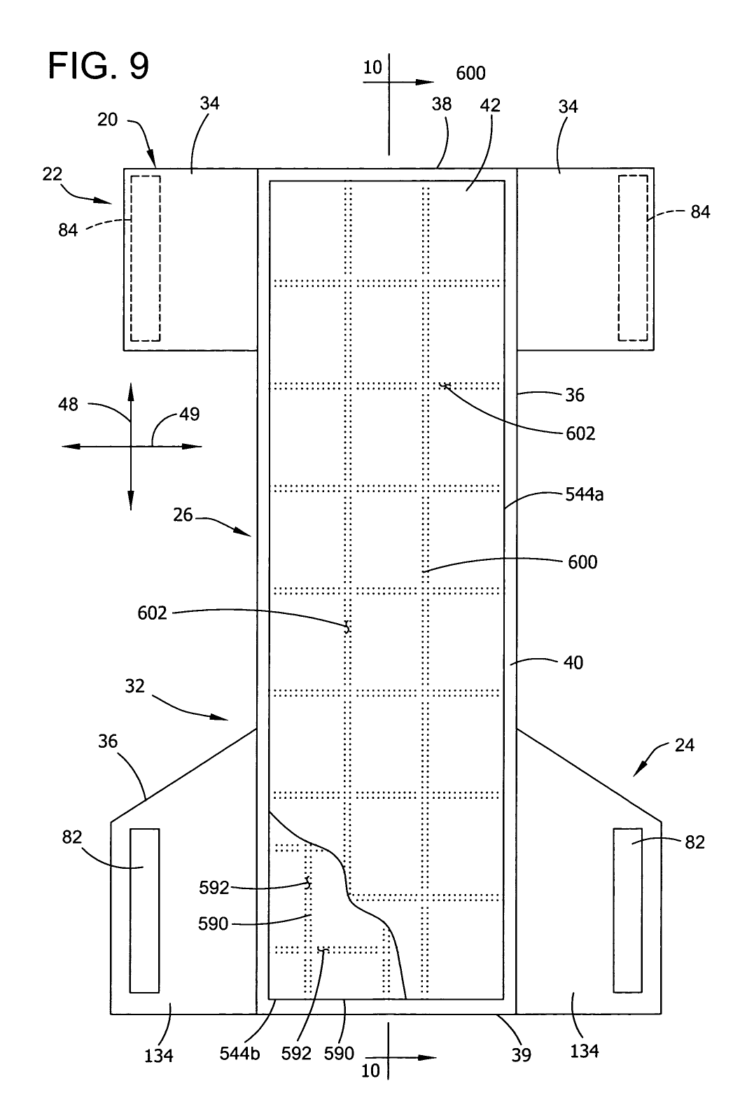

FIG. 9 illustrates an embodiment similar to that of FIGS. 1-3 in that the pants 20 comprises a central absorbent assembly 32 and front and back side panels 34, 134. The central absorbent assembly 32 comprises a liner 42, an outer cover 40 and a pair of absorbent structures 544a, 544b disposed therebetween in superposed relationship, and more particularly overlapping relationship, with each other. The outer absorbent structure 544b suitably comprises a plurality of absorbent segments 590 (e.g., at least two, and in the illustrated embodiment more than two) arranged in generally edge-facing-edge relationship with each other. In this embodiment, boundary regions 592 (e.g., at least one and in the illustrated embodiment a plurality thereof as indicated by the dotted lines in FIGS. 9 and 10) extend between and interconnect adjacent absorbent segments 590 of the outer absorbent structure 544b. The boundary regions 592 are suitably constructed to permit movement adjacent absorbent segments 590 relative to each other, such as flexing at the boundary regions or further separating from each other at the boundary regions, upon movement (e.g., bending or stretching) of the outer cover 40 and/or the liner 42 of the central absorbent assembly 32.

More particularly, the absorbent segments 590 (but not the boundary regions 592) of the outer absorbent structure 544b are suitably attached, such as by adhesive, by ultrasonic bonding or thermal bonding, or by other suitable attachment techniques, to the outer cover 40 for movement with the outer cover. The boundary regions 592 between adjacent absorbent segments are suitably constructed such that upon stretching of the outer cover 40, the absorbent segments move with the outer cover to further separate from each other generally at the boundary regions. For example, in one particularly suitable embodiment, the outer absorbent structure 544b has a density at each boundary region 592 that is substantially less than the density of the outer absorbent structure at each of the absorbent segments 590. In another embodiment, the outer absorbent structure 544b has a basis weight at each boundary region 592 that is substantially less than the basis weight of the outer absorbent structure at each of the absorbent segments 590.

One suitable method of forming such an absorbent structure 544b is to insert an additional wire mesh screen (not shown) over the foraminous forming surface of a conventional air-forming device (not shown). As noted previously, air-forming devices are well known to those skilled in the art for use in forming fibrous webs. In such a device, discrete fibers such as hydrophilic fibers are introduced into the device along with particulate or fibrous superabsorbent material. The absorbent fibers and superabsorbent material are entrained in an air stream within the forming device and directed onto a foraminous forming surface upon which the mixture of absorbent fibers and superabsorbent material is collected to form an absorbent fibrous web or structure.

Air-forming devices employed in high-speed commercial operations typically have a forming surface constructed of a wire screen or fluted grid, and one or more form members which, together with the wire screen or fluted grid, generally define the length, width and thickness profiles of the absorbent structure to be formed on the forming surface. A pneumatic flow mechanism, such as a vacuum suction system, draws the air-entrained fiber stream within the air-forming device toward the forming surface so that air passes through the foraminous surface while the fibers and superabsorbent material collect on the forming surface to form the absorbent structure.

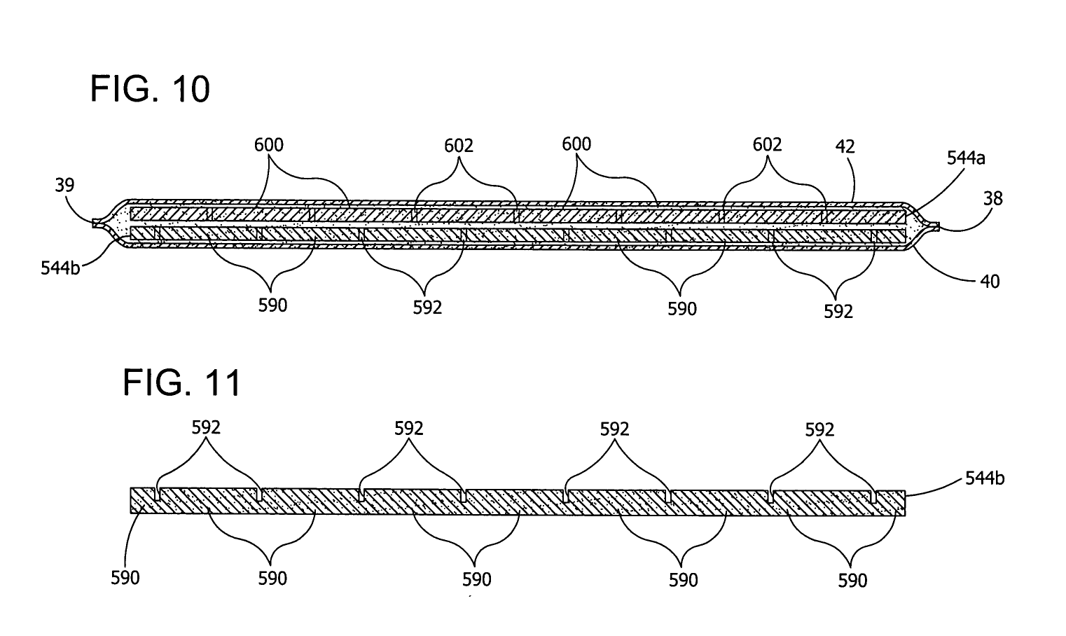

In accordance with one embodiment of a method for making the outer absorbent structure the additional wire screen is placed over the foraminous forming surface to impede the collection of fibers and superabsorbent material on the forming surface at the locations of the wires of the additional wire screen. As fibers and superabsorbent material are collected on the forming surface to form the absorbent structure, a lesser amount of material is collected on the forming surface at the wires of the additional wire screen. The formed absorbent structure 544b thus appears as illustrated in FIG. 11 having absorbent segments 590 interconnected by boundary regions 592 (e.g., where the wires of the additional wire screen were located) whereby the boundary regions have a lower basis weight than the absorbent segments. The absorbent structure 544b may be further processed, such as by passing the absorbent structure through a nip defined by opposed rolls in order to compress the absorbent structure down to a uniform thickness as shown in FIG. 10. Following compression in this manner, the boundary regions 592 of the absorbent structure 544b have a lower density than the absorbent segments of the absorbent structure.

The inner absorbent structure 544a is suitably constructed in substantially the same manner as the outer absorbent structure 544b to comprise a plurality of absorbent segments 600 arranged in edge-facing-edge relationship and interconnected by one or more boundary regions 602 (also indicated by dotted lines in FIGS. 9 and 10). More suitably, the inner absorbent structure 544a is constructed so that the absorbent segments 600 thereof are moveable relative to each other at the boundary regions 602 (e.g., flexible at the boundary regions or further separable from each other at the boundary regions). For example, the inner absorbent structure 544a may be air-formed as described above for the outer absorbent structure 544b. The absorbent segments 600 of the inner absorbent structure 544a are suitably attached to the liner 42 or other substrate of the absorbent assembly 32 as described in previous embodiments. However, it is understood that the absorbent segments 600 of the inner absorbent structure 544a may be free from attachment to any substrate of the absorbent assembly 32 without departing from the scope of this invention.

In the illustrated embodiment of FIG. 9, the absorbent segments 600, 590 and boundary regions 602, 592 of the inner and outer absorbent structures 544a, 544b are configured and arranged relative to each other in substantially the same manner as the absorbent segments 300, 290 and boundary regions 302, 292 of the embodiment shown in FIG. 6. It is contemplated, however, that the absorbent segments 600, 590 and boundary regions 602, 592 of the inner and outer absorbent structures 544a, 544b may be configured and arranged in accordance with any of the embodiments described herein. It is also contemplated that only one of the inner and outer absorbent structures 544a, 544b may comprise interconnected absorbent segments while the other one of the inner and outer absorbent structures may comprise discrete absorbent segments.

It is also understood that throughout the various embodiments shown in the drawings and described herein, only one of the inner and outer absorbent structures may be segmented while the other absorbent structure is non-segmented.

When introducing elements of the present invention or the preferred embodiment(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

1. An absorbent article comprising:
a first substrate;
a second substrate in generally superposed relationship with the first substrate;
a first absorbent structure disposed between the first and second substrates, the first absorbent structure comprising at least two absorbent segments arranged in generally adjacent, edge-facing-edge relationship with each other and having a boundary region between said adjacent absorbent segments; and
a second absorbent structure disposed between the first and second substrates in generally superposed relationship with the first absorbent structure, the second absorbent structure comprising at least two absorbent segments arranged in generally adjacent, edge-facing-edge relationship with each other and having a boundary region between said adjacent absorbent segments, at least one absorbent segment of the second absorbent structure being in superposed relationship with at least a portion of at least two adjacent absorbent segments of the first absorbent structure and the boundary region between said at least two adjacent absorbent segments of the first absorbent structure.

2. An absorbent article as set forth in claim 1 wherein at least one absorbent segment of the first absorbent structure is attached to the first substrate.

3. An absorbent article as set forth in claim 2 wherein each of the absorbent segments of the first absorbent structure is attached to the first substrate.

4. An absorbent article as set forth in claim 2 wherein at least one absorbent segment of the second absorbent structure is attached to the second substrate.

5. An absorbent article as set forth in claim 1 wherein the absorbent segments of the first absorbent structure are free from attachment to the absorbent segments of the second absorbent structure.

6. An absorbent article as set forth in claim 1 wherein at least one of the first and second substrates is stretchable, at least one absorbent segment of the second absorbent structure remaining in superposed relationship with at least two adjacent absorbent segments and the boundary region therebetween of the first absorbent structure upon stretching of at least one of the outer cover and the liner.

7. An absorbent article as set forth in claim 6 wherein adjacent absorbent segments of at least one of the first absorbent structure and the second absorbent structure are separable from each other at the boundary region therebetween upon stretching of said at least one of the first and second substrates.

8. An absorbent article as set forth in claim 6 wherein said at least one of the first and second substrates is elastic.

9. An absorbent article as set forth in claim 1 wherein the absorbent segments of the first absorbent structure are discrete absorbent segments.

10. An absorbent article as set forth in claim 9 wherein the absorbent segments of the second absorbent structure are discrete absorbent segments.

11. An absorbent article as set forth in claim 9 wherein adjacent absorbent segments of the first absorbent structure are spaced from each other at the boundary region therebetween a distance of less than about 5 mm.

12. An absorbent article as set forth in claim 9 wherein adjacent absorbent segments of the first absorbent structure are in abutting relationship with each other at the boundary region therebetween.

13. An absorbent article as set forth in claim 2 wherein at least one absorbent segment of the first absorbent structure has a length and a width and is attached to the first substrate at an attachment region, the attachment region having a length and a width wherein at least one of the length of the attachment region is substantially smaller than the length of the absorbent segment and the width of the attachment region is substantially smaller than the width of the absorbent segment.

14. An absorbent article as set forth in claim 1 wherein adjacent absorbent segments of the first absorbent structure are interconnected at the boundary region therebetween.

15. An absorbent article as set forth in claim 14 wherein the first absorbent structure has a density at each of the adjacent absorbent segments and a density at the boundary region between adjacent absorbent segments which is substantially less than the density of the first absorbent structure at each of the adjacent absorbent segments.

16. An absorbent article as set forth in claim 14 wherein the first absorbent structure has a basis weight at each of the adjacent absorbent segments and a basis weight at the boundary region between adjacent absorbent segments which is substantially less than the basis weight of the first absorbent structure at each of the adjacent absorbent segments.

17. An absorbent article as set forth in claim 1 wherein at least one absorbent segment of the first absorbent structure has one of a different density, a different thickness, a different basis weight, a different length and a different width relative to at least one other absorbent segment of the first absorbent structure.

18. An absorbent article as set forth in claim 1 wherein at least one absorbent segment of the first absorbent structure has one of a different density, a different thickness, a different basis weight, a different length and a different width relative to at least one absorbent segment of the second absorbent structure.

19. An absorbent article as set forth in claim 1 wherein the absorbent segments of the first absorbent structure each comprise a mixture of hydrophilic fibers and superabsorbent material.

20. An absorbent article as set forth in claim 19 wherein the absorbent segments of the second absorbent structure each comprise a mixture of hydrophilic fibers and superabsorbent material.

21. An absorbent article as set forth in claim 19 wherein at least one absorbent segment of the first absorbent structure has a superabsorbent material concentration which is different from a superabsorbent material concentration of at least one other absorbent segment of the first absorbent structure.

22. An absorbent article as set forth in claim 1 wherein the first and second absorbent structure are in generally overlapping relationship with each other whereby at least one absorbent segment of the second absorbent structure is in overlapping relationship with at least a portion of at least two adjacent absorbent segments of the first absorbent structure and the boundary region between said at least two adjacent absorbent segments of the first absorbent structure.

23. An absorbent article as set forth in claim 1 wherein at least one absorbent segment of the first absorbent structure is in superposed relationship with at least a portion of at least two adjacent absorbent segments of the second absorbent structure and the boundary region between said at least two adjacent absorbent segments of the second absorbent structure.

24. An absorbent article as set forth in claim 1 wherein the first absorbent structure comprises at least four absorbent segments arranged in adjacent, edge-facing-edge relationship with each other and having a boundary region between adjacent absorbent segments, at least one absorbent segment of the second absorbent structure being in overlapping relationship with at least a portion of at least four adjacent absorbent segments of the first absorbent structure and the boundary region between said at least four adjacent absorbent segments of the first absorbent structure.

25. An absorbent article as set forth in claim 1 comprising an outer cover defined by the first substrate and a liquid permeable liner defined by the second substrate, the liquid permeable liner being adapted for contiguous relationship with the wearer of the absorbent article.

26. An absorbent article as set forth in claim 1 wherein the absorbent article comprises a pair of training pants.

27. An absorbent article as set forth in claim 1 wherein at least one absorbent segment of the first absorbent structure is oriented to extend in a direction which is different from a direction of extension of at least one other absorbent segment of the first absorbent structure.

28. An absorbent article as set forth in claim 1 wherein at least one absorbent segment of at least one of the first and second absorbent structures comprises a liquid permeable enclosure and at least one absorbent component disposed within the enclosure.

29. An absorbent article as set forth in claim 28 where the absorbent component comprises at least one of hydrophilic fibers and superabsorbent material.

30. An absorbent article comprising:
a first substrate;
a second substrate in generally superposed relationship with the first substrate;
a first absorbent structure disposed between the first and second substrates, the first absorbent structure comprising at least two adjacent absorbent segments arranged in generally edge-facing-edge relationship with each other and having a boundary region therebetween, each of said absorbent segments being attached to the first substrate for movement therewith; and
a second absorbent structure disposed between the first and second substrates in generally superposed relationship with the first absorbent structure, the second absorbent structure comprising at least two adjacent absorbent segments arranged in generally edge-facing-edge relationship with each other and having a boundary region therebetween, each of the absorbent segments of the second absorbent structure being attached to the second substrate for movement with the second substrate, the absorbent segments of the second absorbent structure being free from attachment to the absorbent segments of the first absorbent structure to permit movement of the absorbent segments of the second absorbent structure relative to the absorbent segments of the first absorbent structure upon movement of the first and second substrates.

31. An absorbent article as set forth in claim 30 wherein at least one of the first and second substrates is stretchable.

32. An absorbent article as set forth in claim 31 wherein said at least one of the first and second substrates is elastic.

33. An absorbent article as set forth in claim 30 wherein the absorbent segments of the first absorbent structure are discrete absorbent segments.

34. An absorbent article as set forth in claim 33 wherein the absorbent segments of the second absorbent structure are discrete absorbent segments.

35. An absorbent article as set forth in claim 33 wherein adjacent absorbent segments of the first absorbent structure are spaced from each other at the boundary region therebetween.

36. An absorbent article as set forth in claim 33 wherein adjacent absorbent segments of the first absorbent structure are in abutting relationship with each other at the boundary region therebetween.

37. An absorbent article as set forth in claim 35 wherein the absorbent segments of the second absorbent structure are discrete absorbent segments, said absorbent segments being spaced from each other at the boundary region therebetween.

38. An absorbent article as set forth in claim 31 wherein the first substrate is stretchable, adjacent absorbent segments of the first absorbent structure being separable from each other upon stretching of the first substrate.

39. An absorbent article as set forth in claim 38 wherein the second substrate is also stretchable, adjacent absorbent segments of the second absorbent structure being separable from each other upon stretching of the second substrate.

40. An absorbent article as set forth in claim 31 wherein the second substrate is stretchable, adjacent absorbent segments of the second absorbent structure being separable from each other upon stretching of the second substrate.

41. An absorbent article as set forth in claim 30 wherein adjacent absorbent segments of the first absorbent structure are interconnected at the boundary region between said adjacent absorbent segments.

42. An absorbent article as set forth in claim 41 wherein the first absorbent structure has a density at each of the adjacent absorbent segments and a density at the boundary region between adjacent absorbent segments which is substantially less than the density of the first absorbent structure at each of the adjacent absorbent segments.

43. An absorbent article as set forth in claim 41 wherein the first absorbent structure has a basis weight at each of the adjacent absorbent segments and a basis weight at the boundary region between adjacent absorbent segments which is substantially less than the basis weight of the first absorbent structure at each of the adjacent absorbent segments.

44. An absorbent article as set forth in claim 30 wherein at least one absorbent segment of the first absorbent structure has one of a different density, a different thickness, a different basis weight, a different length and a different width relative to at least one other absorbent segment of the first absorbent structure.

45. An absorbent article as set forth in claim 30 wherein at least one absorbent segment of the first absorbent structure has one of a different density, a different thickness, a different basis weight, a different length and a different width relative to at least one absorbent segment of the second absorbent structure.

46. An absorbent article as set forth in claim 30 wherein the absorbent segments of the first absorbent structure each comprise a mixture of hydrophilic fibers and superabsorbent material.

47. An absorbent article as set forth in claim 46 wherein the absorbent segments of the second absorbent structure each comprise a mixture of hydrophilic fibers and superabsorbent material.

48. An absorbent article as set forth in claim 46 wherein at least one absorbent segment of the first absorbent structure has a superabsorbent material concentration which is different from a superabsorbent material concentration of at least one other absorbent segment of the first absorbent structure.

49. An absorbent article as set forth in claim 47 wherein at least one absorbent segment of the first absorbent structure has a superabsorbent material concentration which is different from a superabsorbent material concentration of at least one absorbent segment of the second absorbent structure.

50. An absorbent article as set forth in claim 30 wherein the absorbent article comprises an outer cover defined by the first substrate and a liquid permeable liner defined by the second substrate and adapted for contiguous relationship with a wearer of the absorbent article.

51. An absorbent article as set forth in claim 30 wherein the absorbent article comprises a pair of training pants.

52. An absorbent article as set forth in claim 30 wherein at least one absorbent segment of at least one of the first and second absorbent structures comprises a liquid permeable enclosure and at least one absorbent component disposed within the enclosure.

53. An absorbent article as set forth in claim 52 where the absorbent component comprises at least one of hydrophilic fibers and superabsorbent material.

54. An absorbent article comprising:
a substrate; and
an absorbent structure comprising at least two adjacent absorbent segments arranged in generally edge-facing-edge relationship with each other and having a boundary region therebetween, the absorbent segments being attached to the substrate for movement with said substrate, said absorbent segments and boundary region being constructed to permit movement of the absorbent segments relative to each other at the boundary region therebetween upon movement of the substrate.

55. An absorbent article as set forth in claim 54 wherein the substrate is stretchable, the absorbent segments being separable from each other at the boundary region therebetween upon stretching of the substrate.

56. An absorbent article as set forth in claim 54 wherein the absorbent segments of the absorbent structure are interconnected at the boundary region therebetween.

57. An absorbent article as set forth in claim 55 wherein the absorbent structure has a density at each absorbent segment and a density at the boundary region which is substantially less than the density of the absorbent structure at each absorbent segment.

58. An absorbent article as set forth in claim 55 wherein the absorbent structure has a basis weight at each absorbent segment and a basis weight at the boundary region which is substantially less than the basis weight of the absorbent structure at each absorbent segment.

59. An absorbent article as set forth in claim 55 wherein the absorbent segments are discrete absorbent segments.

60. An absorbent article as set forth in claim 59 wherein the absorbent segments are spaced from each other at the boundary region therebetween.

61. An absorbent article as set forth in claim 59 wherein the absorbent segments are in abutting relationship at the boundary region therebetween.

62. An absorbent article as set forth in claim 54 wherein the absorbent article comprises an outer cover and a liquid permeable liner in superposed relationship with the outer cover, the absorbent structure being disposed between the liner and the outer cover, one of the liner and the outer cover defining the substrate to which the absorbent segments of the absorbent structure are attached.

63. An absorbent article as set forth in claim 54 wherein at least one absorbent segment of the absorbent structure has one of a different density, a different thickness, a different basis weight, a different length and a different width relative to at least one other absorbent segment of the absorbent structure.

64. An absorbent article as set forth in claim 54 wherein the absorbent segments of the absorbent structure each comprise a mixture of hydrophilic fibers and superabsorbent material.

65. An absorbent article as set forth in claim 64 wherein at least one absorbent segment of the absorbent structure has a superabsorbent material concentration which is different from a superabsorbent material concentration of at least one other absorbent segment of the absorbent structure.
